(19) United States
(12) Patent Application Publication
(10) Pub. No.: US 2011/0318271 A1
(43) Pub. Date: Dec. 29, 2011

(54) Arginase Inhibitors for the Treatment of Depression

(75) Inventors: Vallo Volke, Tartu (EE); Maarja Krass, Tartu (EE); Annika Volke, Tartu (EE); Eero Vasar, Tartu (EE)

(73) Assignee: UNIVERSITY OF TARTU, Tartu (EE)

(21) Appl. No.: 13/141,992

(22) PCT Filed: Dec. 29, 2008
(86) PCT No.: PCT/EE2008/000027

§ 371 Date: Sep. 14, 2011

(51) Int. Cl. A61K 31/197; C12Q 1/34; A61P 25/24; C07C 229/26; A61K 49/00

(52) U.S. Cl. 424/9.2; 562/561; 562/553; 514/564; 514/561; 435/18

(57) ABSTRACT

The subjects of the current invention are compounds, which exhibit arginase inhibiting activity (including difluoromethylornithine (DFMO) and L-norvaline, but not limited to them), and which can be used as therapeutically active agents for the treatment and prevention of depression and/or depression-related conditions. Other subjects of the present invention are the use of said arginase inhibiting compounds as therapeutically active agents for the manufacture of pharmaceutical compositions for human and veterinary application, pharmaceutical composition comprising said arginase inhibiting compound and a method for treatment and prevention of depression and/or depression-related conditions. Also, a method for identifying compounds suitable for use as therapeutically active agents for treatment and/or prevention of depression and/or depression-related conditions is disclosed. The invention further comprises a kit for selecting compounds for treatment and/or prevention of depression and/or depression-related conditions.

### FIELD OF INVENTION

The present invention relates to compositions useful in the therapy of depression and depression-related conditions. More particularly the invention relates to compounds, which exhibit arginase inhibiting activity.

### BACKGROUND OF THE INVENTION

Arginase (EC Nr 3.5.3.1) is an enzyme, activity of which results in converting the amino acid

L-arginine into L-ornithine and urea, being an essential part of the urea cycle. In addition to being metabolized to L-ornithine, L-arginine is also a precursor of NO, a free radical molecule involved in a wide range of biological processes.

Several compounds useful for the treatment of a variety of diseases and disorders have been also shown as possessing arginase inhibiting activity. E.g. arginase inhibitors N(omega)-hydroxy-L-arginine (NOHA), N′-hydroxy-nor-L-arginine (nor-NOHA), 2 (S)-amino-6-boronohexanoic acid (ABH), S-(+)-Amino-6-iodoacetamidohexanoic acid, S-(+)-Amino-5-iodoacetamidopentanoic acid, L-norvaline, or L-HOArg have been shown as a possible means for treatment of asthma, pulmonary hypertension and sickle cell disease (WO/2004/073623 Treatment of conditions associated with decreased nitric oxide bioavailability, including elevated arginase conditions).

L-norvaline is considered to be a potent arginase inhibitor (Rognstad R. Sources of ammonia for urea synthesis in isolated rat liver cells. Biochim Biophys Acta 1977; 496: 249-254; Chang C I, Liao J C, Kuo L. Arginase modulates nitric oxide production in activated macrophages. Am J Physiol 1998; 274: H342-H348). Alpha-DFMO is well-known as an inhibitor of ornithine decarboxylase, but is also a potent inhibitor of arginase (Selamnia M, Mayeur C, Robert V, Blachier F. Alpha-difluoromethylornithine (DFMO) as a potent arginase activity inhibitor in human colon carcinoma cells. Biochem Pharmacol 1998; 55: 1241-1245.3)

The use of alfa-DFMO (eflornithine, sometimes called “eflornithine”) is known and has been described alone or in combination with other compounds in U.S. Pat. No. 6,573,290 for the treatment or prevention of cancer (DFMO and celecoxib in combination for cancer chemoprevention and therapy), U.S. Pat. No. 6,258,845 (DFMO and sulindac combination in cancer chemoprevention) and U.S. Pat. No. 4,925,835 (Aziridinyl putrescine containing compositions and their uses in treating prostate cancer), U.S. Pat. No. 6,277,411 (Pharmaceutical formulation containing DFMO for the treatment of cancer).

Norvaline is an analog of the branched chain amino acid valine and is not present among the 20 common natural amino acid compounds of proteins. It has been used in various combinations in research work being included into peptides, as well as in nutritional compositions, e.g. in WO/2008/067641 (Composition for improving blood flow in working muscles comprising L-arginine, Crataegus extract and artichoke flavonoids).

It has been demonstrated, that increased activity of arginase results in a diminished output of NO (Que L G, George S E, Gotoh T, Mori M, Huang Y C. Effects of arginase isoforms on NO production by nNOS. Nitric Oxide 2002; 6: 1-8.) Thus, arginase inhibitors may increase the production of NO. As NO synthase inhibitors possess antidepressant and anxiolytic like properties (Volke V, Wegener G, Bourin M, Vasar E. Antidepressant- and anxiolytic-like effects of selective neuronal NOS inhibitor 1-(2-trifluoromethylphenyl)-imidazole in mice. Behavioural Brain Research 2003; 140: 141-147), arginase inhibitors should have the opposite effect. Surprisingly, as we demonstrate in the following invention, arginase inhibitors possess antidepressant properties.

Hereby a new potent and surprising effect of two structurally distinct arginase inhibitors DFMO and L-norvaline has been disclosed leading to the perspective to use compounds with arginase inhibiting activity in the treatment and/or prevention of depression and/or depression-related conditions.

### BRIEF DESCRIPTION OF THE DRAWINGS

FIG. 1 Effect of imipramine (15 mg/kg), L-norvaline, and DFMO in the forced swimming test (n=8 per group). Results are expressed as mean±S.E.M. *—P<0.01, **—P<0.001, versus saline.

FIG. 2 Effect of L-ornithine (500 mg/kg), L-norvaline (500 mg/kg), and their combination in the forced swimming test (n=10 per group). Results are expressed as mean±S.E.M. *—P<0.01 vs. saline+saline group; #—P<0.001, vs. Sal+L-norvaline group.

### DISCLOSURE OF THE INVENTION

It has now been found that arginase inhibitors, e.g., L-norvaline and alpha-DFMO are therapeutically promising as antidepressant agents.

The subjects of the current invention are compounds, which exhibit arginase inhibiting activity (including difluoromethylornithine (DFMO) and L-norvaline, but not limited to them), and which therefore can be used as therapeutically active agents for the treatment and prevention of depression and/or depression-related conditions.

The compounds inhibiting arginase activity can be difluoromethylornithine (DFMO), L-norvaline, but not limited to them.

Other subjects of the present invention are: the use of said arginase inhibiting compounds as therapeutically active agents for the manufacture of pharmaceutical compositions for human and veterinary application; and pharmaceutical compositions comprising said compounds and pharmaceutically acceptable carrier. Representatives of such carriers are generally known in the human and veterinary pharmaceutical field. Examples of such carriers are starch, alginates, stearates, gelatin, lactose, microcrystalline cellulose, etc. The pharmaceutical compositions may have any form suitable for its application, for instance they may be in the form of capsule, powder, tablet, solution, suspension, lotion, etc.

Thus, difluoromethylornithine (DFMO), L-norvaline or other arginase inhibitors can be used as therapeutically active agents for manufacturing pharmaceutical compositions for human and veterinary application. The pharmaceutical compositions comprising difluoromethylornithine (DFMO), L-norvaline or other arginase inhibitors can be used for treatment and/or prevention of depression and/or depression-related conditions.

Also, a method for identifying compounds suitable for use as therapeutically active agents for treatment and/or prevention of depression and/or depression-related conditions and/or anxiety is disclosed, which comprises determining whether the compound under investigation exhibits arginase inhibiting activity, and if the named compound exhibits arginase inhibiting activity, then the named compound is selected as candidate for a compound suitable for use as therapeutically active agent for treatment and/or prevention the named diseases and/or conditions.

Also included in the invention is a method for treatment and prevention of depression and/or depression-related conditions, which comprises administering to a mammal suffering from depression and/or depression-related conditions or a mammal supposed to gain depression or depression related disorder a therapeutically effective amount of a compound which exhibits arginase inhibiting activity.

Moreover, a kit for selecting novel compounds for treatment and/or prevention of depression and/or depression-related conditions is disclosed. This kind of kit comprises at least a means for determining a compound's arginase inhibiting activity by a method known to the person skilled in the art. If arginase inhibiting activity is detected, then the compound is selected for a further screening of its properties as a drug candidate for the treatment and/or prevention of depression and/or depression-related conditions.

### DESCRIPTION OF EMBODIMENTS

The antidepressant properties of arginase inhibiting agents L-norvaline and DFMO were investigated by means of a set of behavioural tests in animals conventionally employed in pharmacology and generally considered predictive of antidepressant activity in man (Porsolt R D, Bertin A, Jalfre M. Behavioral despair in mice: a primary screening test for antidepressants. Arch Int Pharmacodyn Ther 1977; 229: 327-336; Crawley J, Goodwin F K. L-norvaline, DFMO and L-ornithine have been tested in the following tests: Forced Swimming Test in mice, (Example 1, Example 2), Locomotor Activity Test (Example 3).

Locomotor activity of animals was measured using an automated system as described previously (Volke V, Wegener G, Bourin M, Vasar E. Antidepressant- and anxiolytic-like effects of selective neuronal NOS inhibitor 1-(2-trifluoromethylphenyl)-imidazole in mice. Behavioural Brain Research 2003; 140: 141-147).

Data were analyzed with one-way or two-way analysis of variance (ANOVA) when appropriate. Post hoc comparisons between individual groups were performed by Newman-Keuls test.

### EXAMPLE 1

### Forced Swimming Test

Male C57BL/6/Bkl mice (Scanbur BK AB, Sweden) weighing 20-25 g were used. Mice were kept 10 per cage (21×37×15 cm) in an animal house at 20° C. in a 12 h light/dark cycle (light on at 7.00 a.m.). Tap water and food pellets were available ad libitum. The animals were kept for at least two weeks in the animal colony before entering experiments.

The measurement of locomotor activity, and forced swimming test were carried out consecutively 45 and 55 min after treatment with study compounds, according to Volke V, Wegener G, Bourin M, Vasar E. Antidepressant- and anxiolytic-like effects of selective neuronal NOS inhibitor 1-(2-trifluoromethylphenyl)-imidazole in mice. Behavioural Brain Research 2003; 140: 141-147. In a separate experiment arginase product L-ornithine or saline was injected 15 min prior to L-norvaline.

The Forced Swimming test was performed as described by Porsolt et al Porsolt R D, Bertin A, Jalfre M. Behavioral despair in mice: a primary screening test for antidepressants. Arch Int Pharmacodyn Ther 1977; 229: 327-336.

A glass cylinder with a diameter of 12 cm was filled with 8 cm water at 25° C. An animal was put into the water and its behaviour was videotaped during 6 min. An observer blind to treatment protocol counted the immobility time during the last 4 min of the 6 min test.

### Results:

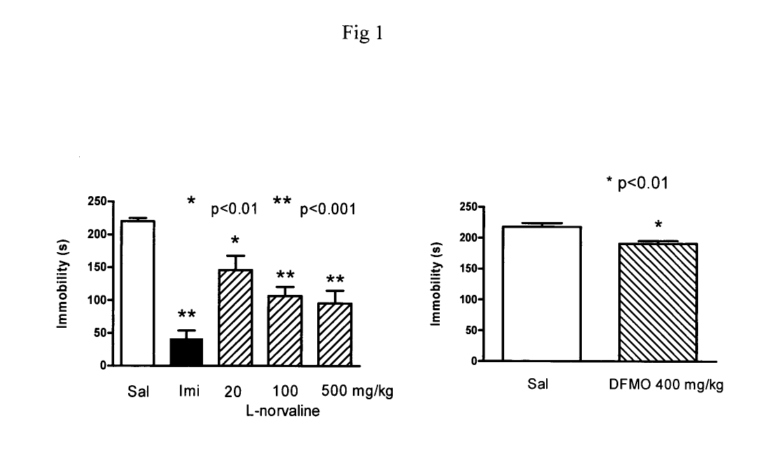

The immobility time was 220 sec in control animals. L-norvaline given intraperitoneally (i.p.) in the doses of 20, 100 and 500 mg/kg decreased the immobility time to 146, 107 and 96 sec, respectively (FIG. 1).

In a separate experiment DFMO (400 mg/kg, i.p.) decreased the immobility time from 219 sec to 191 sec. Lower doses of DFMO (10-100 mg/kg, i.p.) were ineffective.

Both L-norvaline and DFMO reduce the immobility time, thus exhibiting an antidepressant-like effect.

### EXAMPLE 2

### Effect of L-ornithine, L-norvaline, and Their Combination in Forced Swimming Test

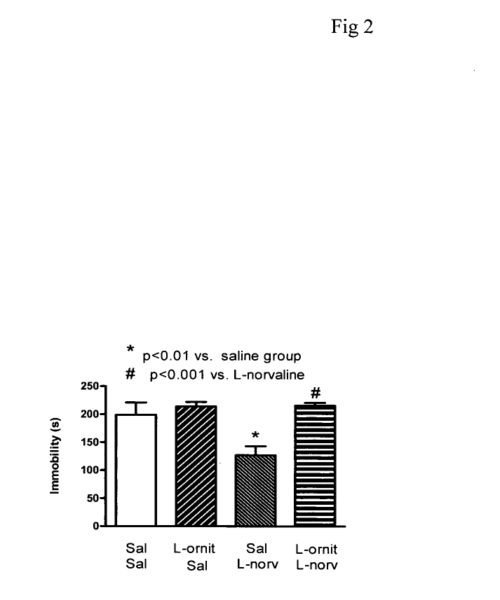

In a separate experiment L-ornithine (500 mg/kg) did not influence the immobility time (saline 199 sec, L-ornithine 214 sec, FIG. 2). However, pre-treatment with L-ornithine antagonised the effect of L-norvaline on immobility time (L-norvaline 127 sec, L-ornithine +L-norvaline 215 sec; p<0.001 vs. L-norvaline group).

Thus, two structurally distinct arginase inhibitors induced antidepressant-like effect, indicating that both molecules, as well as arginase inhibitors in general, are of interest as therapeutic agents in the treatment and/or prevention of depression and/or depression-related conditions.

Moreover, the arginase product L-ornithine was able to block the effect of L-norvaline, further supporting that the antidepressant effect of study compounds are depending on arginase inhibition.

### EXAMPLE 3

### Effect of L-ornithine, L-norvaline, and Their Combination on locomotor Activity

Locomotor activity was measured using an automated system with six chambers (45×45×45 cm) made from transparent acrylic (MOTI, Technical & Scientific Equipment GMBH, Germany; Volke V, Wegener G, Bourin M, Vasar E. Antidepressant- and anxiolytic-like effects of selective neuronal NOS inhibitor 1-(2-trifluoromethylphenyl)-imidazole in mice. Behavioural Brain Research 2003; 140: 141-147). The apparatus-naïve mice were put into the chamber, and vertical and horizontal activity was counted during a 10 min. test period.

### Results:

The study compounds either alone or in combination did not change the ambulation of animals, excluding the possibility that the drug effects in the forced swimming test were of non-specific origin.

The descriptions above lead to the conclusion that compounds exposing arginase inhibiting activity can be used as pharmacological agents for treating and/or preventing of depression and/or depression-related conditions. Difluoromethylornithine (DFMO) has been shown as a candidate for the treatment and/or prevention of depression and/or depression-related conditions. L-norvaline has been shown as a candidate for the treatment and/or prevention of depression and/or depression-related conditions. Accordingly, L-norvaline, DFMO and other compounds with arginase inhibiting activity can be considered as candidates for producing a human medicine for treatment and/or prevention of depression and/or depression-related conditions. Respectively, L-norvaline, DFMO and other compounds with arginase inhibiting activity can be considered as candidates for producing a veterinary medicine for treatment and/or prevention of depression and/or depression-related conditions.

A method for treating or preventing depression and/or depression-related conditions by administering a mammal a pharmaceutical composition comprising a compound, which exhibits arginase inhibiting activity is hereby disclosed.

It is now obvious for one skilled in the art, that other compounds, which exhibit arginase inhibiting activity, may also be useful for treatment and/or prevention of depression and/or depression-related conditions. Thus, a method for identifying a compound suitable for treatment and/or prevention of depression and/or depression-related conditions comprises determining whether the compound under investigation exhibits arginase inhibiting activity, and if the named compound exhibits arginase inhibiting activity, then the named compound is selected as candidate for a compound suitable for the treatment and/or prevention the named diseases and/or conditions.

One skilled in the art can also conclude, that a kit for selecting novel compounds for treatment and/or prevention of depression and/or depression-related conditions comprises at least a means for determining a compound's arginase inhibiting activity by a method known to a person skilled in the art. This means involves a biochemical assay, wherein the production of

L-ornithine or urea is measured, but is not limited to. The assay is performed in vitro on purified enzyme arginase, or in a cell culture or in another model system. If arginase inhibiting activity is detected, then the compound is selected for a further screening of its properties as a drug candidate for the treatment and/or prevention of depression and/or depression-related conditions.

1. Compounds, which exhibit arginase inhibiting activity for use as therapeutically active agents for treatment and/or prevention of depression and/or depression-related conditions.

2. Compound according to claim 1, which is difluoromethylomithine (DFMO).

3. Compound according to claim 1, which is L-norvaline.

4. Use of a compound according to claim 1 as therapeutically active agent for manufacturing a pharmaceutical composition for human application.

5. Use of a compound according to claim 1 as therapeutically active agent for manufacturing a pharmaceutical composition for veterinary application.

6. A pharmaceutical composition for treatment and/or prevention of depression and/or depression-related conditions comprising a therapeutically active agent and a pharmaceutically acceptable carrier, characterized in that the therapeutically active agent is a compound according to claim 1.

7. Method for identifying compounds for use as therapeutically active agents for treatment and/or prevention of depression and/or depression-related conditions comprising a) determining whether the compound under investigation exhibits arginase inhibiting activity, b) and if the named compound exhibits arginase inhibiting activity, then the named compound is identified as a compound applicable for use as therapeutically active agent for treatment and/or prevention of depression and/or depression-related conditions.

8. Method for treating and/or preventing of depression and/or depression-related conditions in a mammal in need of such treatment and/or prevention, said method comprising administering to said mammal a therapeutically effective amount of a compound which exhibits arginase inhibiting activity.

9. Method according to claim 8, whereas said compound is difluoromethylornithine (DFMO).

10. Method according to claim 8, whereas said compound is L-norvaline.

11. A kit for identifying compounds for use as therapeutically active agents for treatment and/or prevention of depression and/or depression-related conditions, which comprises at least a means for determining a compound's arginase inhibiting activity.
